(19) United States
(12) Patent Application Publication
(10) Pub. No.: US 2024/0082037 A1
(43) Pub. Date: Mar. 14, 2024

(54) ORTHOSIS FOR CLUBFOOT (CTEV) CORRECTION AND MAINTENANCE BASED ON PONSITI METHOD

(71) Applicant: JAIPUR CLUB FOOT PRIVATE LIMITED, Jaipur (IN)

(72) Inventor: Harlal Singh Mali, Jaipur (IN)

(21) Appl. No.: 18/272,822

(22) PCT Filed: Dec. 29, 2021
(86) PCT No.: PCT/IN2021/051212

§ 371 Date: Jul. 18, 2023

(30) Foreign Application Priority Data
Jan. 23, 2021 (IN) 202111003245

(51) Int. Cl. A61F 5/01

(52) U.S. Cl. CPC A61F 5/0127; A61F 2005/0137; A61F 2005/0146; A61F 2005/0179

(57) ABSTRACT

This invention relates to Orthosis for clubfoot (CTEV) Correction and Maintenance based on Ponsiti Method.

CTEV (clubfoot) is a congenital deformity prevalent to one in 800 children born. Very few attempts have been made to correct and maintain CTEV deformity by unilateral orthosis, which are associated with drawbacks and hence not effective.

The Orthosis comprising metatarsal top (1), metatarsal bottom (2) and pitch and yaw link (3) connected together by an actuator A1 (13) with the pitch and yaw link (3) placed between the metatarsal top and metatarsal bottom, wherein the metatarsal top (1) and metatarsal bottom (2) are provided with strap slots (1b,2b) for holding baby foot;

The pitch and yaw link (3) is connected to forefoot roll link (4). The forefoot roll link (4) is connected to upper and top bases of hind foot assembly (5, 6) to execute the rolling motion of the forefoot.

### FIELD OF INVENTION

This invention relates to Orthosis for clubfoot (CTEV) Correction and Maintenance based on Ponsiti Method.

The orthosis involves eight motions, out of which first six are corrective motions, three at forefoot level and three at hind foot level and out of the other two motions, one caters to the growth of leg of the patient and another one knee movement of the patient.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Clubfoot, mainly CTEV (Congenital Talipes Equino Varus), is a complex three-dimensional deformity, challenging physicians from ancient times. Among thousand babies born, 1-6 suffers from clubfoot deformity i.e. in India alone, around 35,000 needs treatment for clubfoot annually. 80% of the cases of Clubfoot are from Lower and Middle-Income Countries (LMICs). Among the children affected with clubfoot, 50% of cases are of bilateral deformity i.e. both feet are affected.

### Current CTEV Treatment: Ponseti Method

The most popular, widely accepted and successful method for clubfoot treatment is developed by Dr Ponseti and his method is known as the Ponseti Method. In this method, the deformity is corrected by a series of manipulations through castings by the clinicians followed by maintenance using a bilateral FAB (Foot Abduction Brace). Manipulations are based on the understanding of the biomechanics of the foot. During correction, with the lateral aspect of talus head as fulcrum, the foot follows its natural path in pronation as a closed kinematic chain comprising of forefoot and hindfoot, as shown in FIG. 1. This technique involves the simultaneous correction of all deformities except equinus. After the manipulations by the clinician (FIG. 1), the cast is applied to hold the CTEV affected foot in the corrected position.

The first manipulation is to correct the cavus deformity. For correcting this the following steps are carried out as shown in FIG. 2:
i. Stabilizing the Talus by placing the thumb over the lateral part of the head of the Talus.
ii. Elevating the first ray and achieving a homogeny Supination of the forefoot in line with the midfoot, then abducting the foot gently.
iii. Holding the corrected position while an assistant applies padding and plaster. Changing hand positions and moulding well at the heel, Malleoli and sole of the foot.

In small babies correction of the Cavus usually occurs with the first cast, whereas, a severe Cavus in a stiff foot (older babies) will need 2 or 3 cast changes for correction.

After achieving normal arch through cavus correction, correction of midfoot Inversion and Heel Varus is carried out through successive casts as shown in FIG. 3.

In the “younger child” (i.e. not walked before treatment) the aim is for 50°-60° abduction, and the heel in the neutral position. In the “older child” (i.e. already walked before treatment) aim is for 30°-50° abduction, and the heel in the neutral position.

The procedure is as follows:
i. Stabilizing the Talus by placing the thumb over the lateral part of the head of the Talus.
ii. Holding the foot in Abduction with the appropriate degree of Supination while applying the cast. Supination decreases with the increase of Abduction. At about 50° Abduction, no Supination needs to be kept anymore, but the foot is never to be held in Pronation.
iii. The correction of the Calcaneus is achieved because of coupling. Plaster should be done as often possible to get enough Abduction.
iv. The corrected position is held by the clinician, while an assistant applies padding and plaster. Moulding is done affectively by changing hand positions.

When the desired abduction is achieved, the next step is the correction of rigid equinus in the ankle. This requires Achilles tendon tenotomy (minor surgical procedure) in most cases. After that, plaster is applied for 3 weeks to maintain the foot in the corrected position through the following procedure:
i. Stabilizing the Talus by placing the thumb over the lateral part of the head of the Talus.
ii. Moving the foot in maximal possible Abduction without any Pronation and then into Dorsiflexion in the ankle with the lower hand. By abducting, the Navicular should now fully cover the head of the Talus, so that the thumb will lose the ability to palpate the lateral head of the Talus. Therefore thumb is removed which stabilized the Talus in the beginning, and then moving the foot in Dorsiflexion. To achieve a better Dorsiflexion, grabbing the heel, pulling it down and giving gentle pressure under the entire sole into Dorsiflexion is advisable. Giving mere force or pressure in the forefoot area trying to achieve more Dorsiflexion is never to be done, as it can cause a rocker bottom deformity. After maximal Abduction and Dorsiflexion has been achieved, moving the lower hand to the toes of the child is advisable before casting.
iii. Clinician holds the corrected position while an assistant applies padding and plaster. Moulding is done effectively at the heel, Malleoli and sole of the foot by changing hand positions.

After the cast is removed, the foot may appear overcorrected, but “Hyper abduction” of 70° in babies is a helpful measure to prevent a recurrence.

After the correction is achieved, the maintenance phase starts. In the maintenance phase as shown in FIG. 3(b), Foot Abduction Orthosis/Brace (FAO/FAB) is recommended for 23 hours per day initially up to 3 months. In the case of unilateral deformity, the corrected foot is placed at 70 degree and normal foot at 40-degree abduction whereas in bilateral cases both the feet are in 70-degree abduction. After 3 months, it is recommended to use boots in daytime and FABs in night time for up to 3 years for maintenance of the correction achieved through castings.

### Limitation of Ponseti Method:

Various studies establish that the Ponseti method is a gold standard in clubfoot treatment for 50 years. However, this method suffers from the issues like multiple visits to clinicians, excessive travel requirement at the patient end, longer involvement of clinicians, high dependency on the skill levels of the clinicians, skin ulcers on patients and minimal contemporary technological intervention in treatment etc., which leads to non-start/discontinuation of the treatment, leading to lifelong permanent deformity in the patient, requiring a complex surgical procedure. Almost 50% of the children with clubfoot deformity are left untreated due to these challenges. The bilateral FAB (Foot abduction brace), even if the deformity in unilateral is a social stigma to the patient and family, such perceptions even had led to impairing the patient's natural growth.

### Prior Art of the Invention:

Few un-successful attempts have been made to correct and maintain CTEV deformity by the unilateral orthosis. Faraj et al [1] tested an orthosis orthosis called “Clubax” for clubfoot treatment. The study was carried out from April 1996 to September 1998. The corrective orthosis was applied on 14 patients (21 feet) with an average period of 3 months (varying between 2-4 months). The follow-up period was from 5-32 months with an average of 15 months. Lugo et al [2] developed another corrective orthosis. The corrective orthosis design allowed 4 degrees of freedom (DOF). The orthosis was tested on a unilateral clubfoot in a single patient for 8 months. Kanwaljit et al [3] developed a 3 DOF based orthosis and tested it on 5 patients for one week. Mohamaddan et al [4], developed adjustable foot corrective device for clubfoot treatment. Vishnu et al [5], attempted designing and developing of orthosis for clubfoot correction in infants an additive manufacturing approach.

However, none of the above attempts made success in CTEV treatment and main reason could be attributed to them not following the foot biomechanics as proposed by Dr. Ponsiti. In low and middle income countries no further studies are available in literature for the utility of these orthoses, where such devices are needed the most. In this regard Mali H S, et al. [6] attempted developing a remedial orthosis for CTEV (clubfoot) patient through five corrective motions. However, further development is required, especially in line with Ponsiti's understanding of foot bio-mechanics.

Based on the foot biomechanics understood by Dr. Ponseti, 50 years back, an orthosis based on state of the art technological intervention is needed to be developed which not only overcomes the above problems but also enhances the CTEV treatment experience at all stake holder's level, i.e. the patient, parents, clinician and the society. Hence, the present invention has been introduced, that serves the purpose effectively.

### OBJECTS OF THE INVENTION

An object of the invention is to provide Orthosis for clubfoot (CTEV) Correction and Maintenance based on Ponsiti Method, which obviates shortcomings of the prior art(s).

Still another object of the invention is to provide Orthosis for clubfoot (CTEV) Correction and Maintenance based on Ponsiti Method, which is simple in construction.

Yet another object of the invention is to provide Orthosis for clubfoot (CTEV) Correction and Maintenance based on Ponsiti Method, which effectively serves the purpose.

Yet another object of the invention is to provide Orthosis for clubfoot (CTEV) Correction and Maintenance based on Ponsiti Method, which incorporates the contemporary and futuristic technologies.

These and other objects and advantages of the present invention will be apparent to those skilled in the art after a consideration of the following detailed description taken in conjunction with the accompanying drawings in which a preferred form of the present invention is illustrated.

### SUMMARY OF THE INVENTION

One or more drawbacks of conventional systems and process are overcome, and additional advantages are provided through the apparatus/composition and a method as claimed in the present disclosure. Additional features and advantages are realized through the technicalities of the present disclosure. Other embodiments and aspects of the disclosure are described in detail herein and are considered to be part of the claimed disclosure.

According to the invention, there is provided Orthosis for clubfoot (CTEV) Correction and Maintenance based on Ponsiti Method comprising a metatarsal top (1), a metatarsal bottom (2) and a pitch and yaw link (3) connected together by an actuator A1 (13) with the pitch and yaw link (3) placed between the metatarsal top (1) and metatarsal bottom (2), wherein the metatarsal top (1) and metatarsal bottom (2) are provided with strap slots (1b,2b) allowing passage of strap through the slots (1b,2b) for holding baby foot;
the pitch and yaw link (3) is connected to forefoot roll link (4);
the forefoot roll link (4) is connected to upper and top bases of hind foot assembly (5, 6) to execute the rolling motion of the forefoot;
Yaw Guide Base (7) has a rotating joint with the Hind foot Top Base (6) to provide rolling of the hind foot.
the Yaw Guide Base (7) slides on Hind foot Yaw Link (8) to provide yawing of the hind foot.
the hindfoot yaw link (8) is suspended on calf support links (9) on two sides;
Calf Muscle Support (10) is provided to support the calf muscle and is attached to the Calf Support Links (9).

The Metatarsal Top Link (1) is superimposed on Metatarsal Bottom Link (2), each of which is provided with a slot (1b and 2b) respectively so as to hold foot, wherein the metatarsal top and bottom (1, 2) are coupled with pitch and yaw link (3) supporting the metatarsals to perform the forefoot yawing motion buoyed by the actuator A1 (13) through the hole (3a), in which the actuator A1 (13) passes through the holes (1a, 2a,3a).

The pitch and yaw link (3) is held in place and connected to the forefoot roll link (4) by actuator A2 (14) passing through the keyholes on both the linkages (3b and 4a) respectively.

The Forefoot Roll Link (4) is having rotating joint with Pitch and Jaw Link (3) which gives the pitching motion of the forefoot through actuator A2 (14), in which the Hindfoot Top Base (6) is secured with Hindfoot Bottom Base (5) and the Forefoot Roll Link (4) having joint with both bottom and top base (5,6) to provide rolling of the forefoot using actuator A3 (15), wherein the Hindfoot Top Base (6), Hindfoot Bottom Base (5) and Forefoot Roll Link (4) are connected to each other via said actuator A3 (15).

The hind foot assembly encompasses hind foot lower base (5), hind foot top base (6) and a yaw guide base (7), in which the hind foot top base (6) is provided to support the talus.

The Yaw Guide Base (7) has a rotating joint with the Hindfoot Top Base (6) to provide rolling of the hindfoot through actuator A4 (16).

The Yaw Guide Base (7) slides on the Hindfoot Yaw Link (8) to provide yawing of the hindfoot through actuator A4 (16).

The hindfoot yaw link (8) is suspended on calf support links (9) on two sides by actuator A5 (17) through holes on both the links (8a, 9d) respectively to accomplish the hindfoot pitching motion.

The Calf Muscle Support (10) is provided to support the calf muscle and is fixed to the Calf Support Links (9) using fastener including Bolt (18) passing through holes (9a & 10a). Multitude of holes (9b) are provided on the calf support link (9) at different levels/heights to be locked with the slider link (11) using a slider bolt (19), in which the slider link (11) has a pair of embedded nuts and bolt holes (11a, 11b) for the same. The calf support link has slots (9e, 90 to draw straps there through to tie around the leg.

The Slider Links (11) are attached to Calf Support Links (9) having a plurality of holes (9b), which allows the Slider Links (11) to have linear motion inside Calf Support Links (9) to compensate for the growth of the leg during the treatment.

The Calf Muscle Support (10) supports the calf muscle, which is having protrusions towards the right and left with holes (10a) therein.

The orthosis includes thigh Links (12) for attaching and grounding of the orthosis with the patient thigh using straps passing through slot (12a), wherein the Thigh Links (12) replicate the above-knee part of the cast and the Thigh Links (12) are connected with two Slider Links (11) through a rotating joint, for which a fastener including Bolt (18) passes between two holes (11a and 12b).

The thigh link (12) has a hole (12b) to attach the slider link (11) on one side using a slider bolt (19), that supports the thigh even when the knees are bent, in which the thigh link (12) has a pair of slots (12a) for straps to be secured around the thigh.

The orthosis involves eight motions, out of which first six are corrective motions, three at forefoot level and three at hind foot level and out of the other two motions, one caters to the growth of leg of the patient and another one knee movement of the patient.

The mechanism relies on indexing the rotary motion and locking and unlocking it using splines created on outer body (24), inner slider (21) and outer cap (20), in which the slider in its initial position rests midway between outer cap (20) and outer body (24) making the splines interlock the outer cap (20) and outer body (24) connected to components that rotate relative to one another, wherein upon activation of electromagnet marked (22) the permanent magnet marked (25) attached permanently to inner body (21) is repelled towards the outer cap (20) allowing for rotation of outer cap (20) facilitated by motor (23), wherein once the required amount of rotation is achieved, the electromagnet (22) is deactivated and the spring (26), attached to both outer cap (20) and inner slider (21) restores the inner slider (21) returns to its position of rest locking the mechanism in place till manipulation is required.

The lock and unlock provisions along with dial gauges to all the six corrective motion mechanisms permits the clinicians to manipulate and hold various parts of the foot for successive corrections.

In case of manual operation an Allan key is inserted into the cavity in the outer cap that pushes onto the inner slider unlocking the movement as it slides away from the outer cap allowing for required rotation and then restored to its original position as the spring (26) contracts bringing the inner slider (21) back to its position of rest locking it in place when the key is removed.

The corrective orthosis has provision of close loop actuator along with sensors for step motion as well as locking/unlocking mechanism.

The close loop actuators along with sensors helps in data recording by the controller cum data acquisition system and hence deformity measurement and database creation before, during and after the treatment of the patient can be automated.

Various objects, features, aspects, and advantages of the inventive subject matter will become more apparent from the following detailed description of preferred embodiments, along with the accompanying drawing figures in which like numerals represent like components.

It is to be understood that the aspects and embodiments of the disclosure described above may be used in any combination with each other. Several of the aspects and embodiments may be combined to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The illustrated embodiments of the subject matter will be best understood by reference to the drawings, wherein like parts are designated by like numerals throughout. The following description is intended only by way of example, and simply illustrates certain selected embodiments of devices, systems, and processes that are consistent with the subject matter as claimed herein, wherein: —

FIG. 1 shows: Ponseti method manipulation technique.

FIG. 2 collectively shows: Ponseti method for clubfoot 1s t cast in different views in reference planes.

FIG. 2(a) shows: Frontal view of clubfoot 1s t cast.

FIG. 2(b) shows: Medial (Sagittal) view of clubfoot 1s t cast.

FIG. 2(c) shows: Caudal (Anterior) view of clubfoot 1s t cast.

FIG. 2(d) shows: Reference planes of clubfoot P t cast indicating the following:
Abduction (Yawing-in)—27,
Dorsiflexion (Pitching-up)—28,
Eversion (Rolling-CW from-Front)—29,
Abduction (Yawing-out)—30,
Plantarflexion (Pitching-down)—31,
Inversion (Rolling-CCW from Front)—32

FIG. 3(a) shows: 2^{nd} to 4^{th} casts in Ponseti method for clubfoot adduction and varus correction.

FIG. 3(b) shows: Foot Abduction Orthosis/Brace (FAO/FAB) used during maintenance phase of Ponsiti Method.

FIG. 4 collectively shows: parts of Ponseti Method based Orthosis for CTEV (clubfoot) Correction and Maintenance according to the present invention.

FIG. 4(a) shows: the side view of the CTEV Orthosis.

FIG. 4(b) shows: the rear view of the CTEV Orthosis.

FIG. 4(c) shows: the cross-sectional top view (plane C-C from FIG. 4(a)) of the CTEV Orthosis.

FIG. 4(d) shows: the sectional front view (plane D-D from FIG. 4(a)) of actuator 1 (14) between the pitch and yaw link (3) and forefoot Roll Link (4).

FIG. 4(e) shows: the Top view of Metatarsal Top (1) with hole (1a) and cut section (1b).

FIG. 4(f) shows: the Top view of Metatarsal Bottom (2) with dial gauge, hole (2a) and cut section (2b).

FIG. 4(g) shows: the Top view and side view of Pitch & Yaw link (3) with holes (3a and 3b).

FIG. 4(h) shows: the Side and Back View of Forefoot Roll link (4) with holes (4a and 4b).

FIG. 4(i) shows: the Front view of Hindfoot Lower Base (5) with cut section (5a).

FIG. 46) shows: the Front and side view of Hindfoot Top Base (6) with hole (6a) and cut sections (6b and 6c).

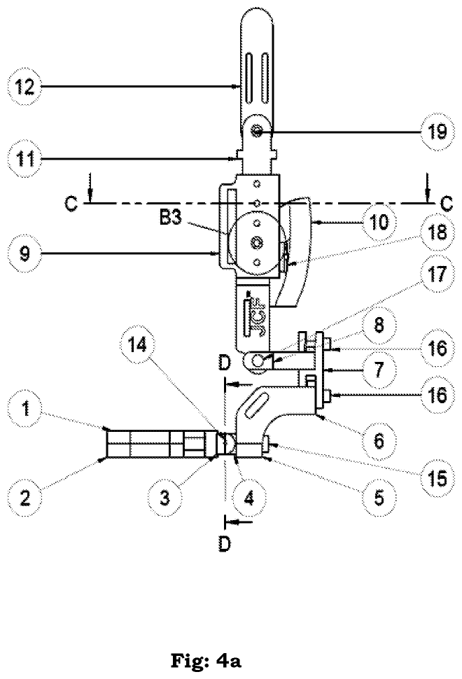

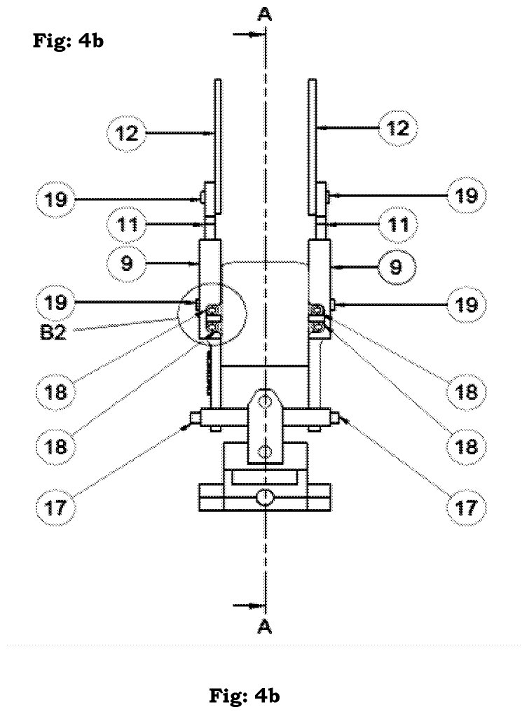

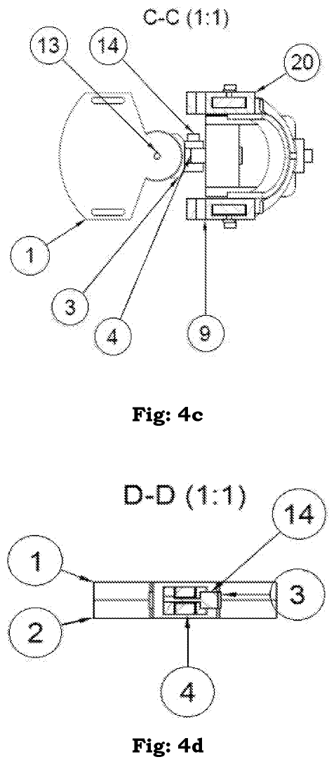

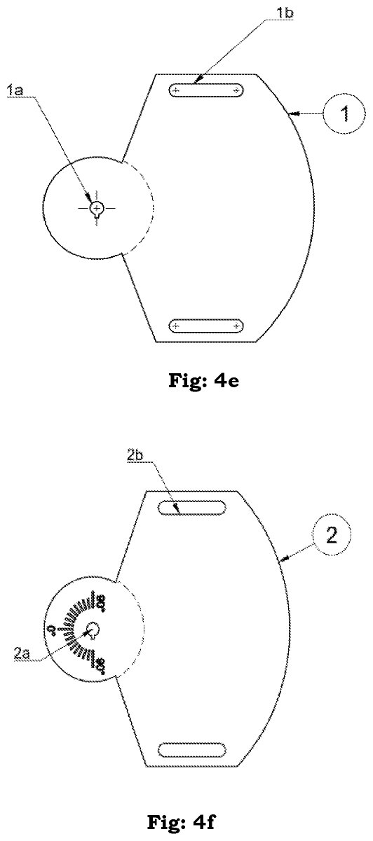

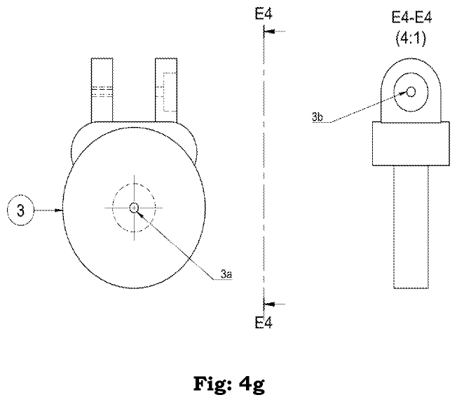

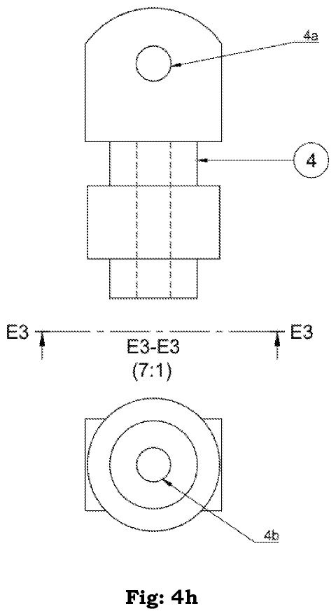

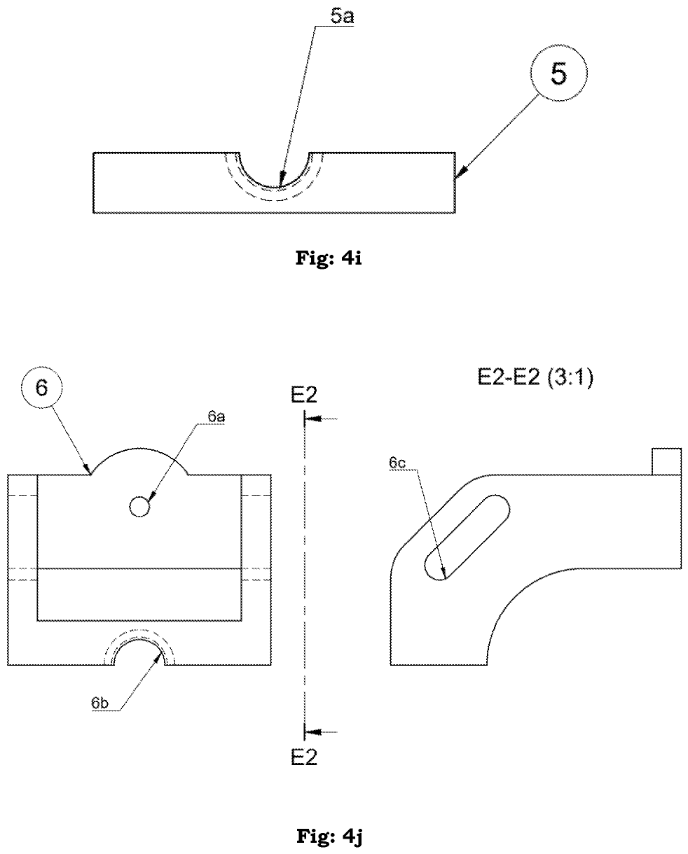

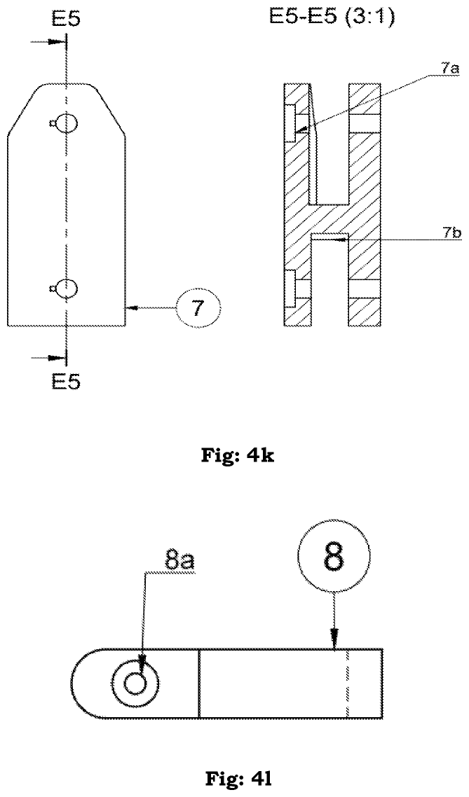

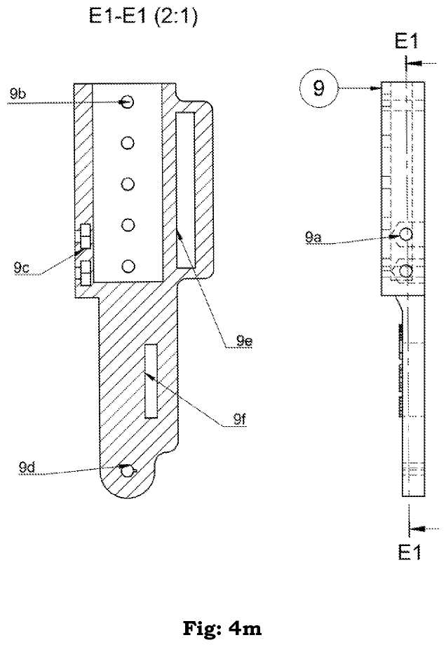

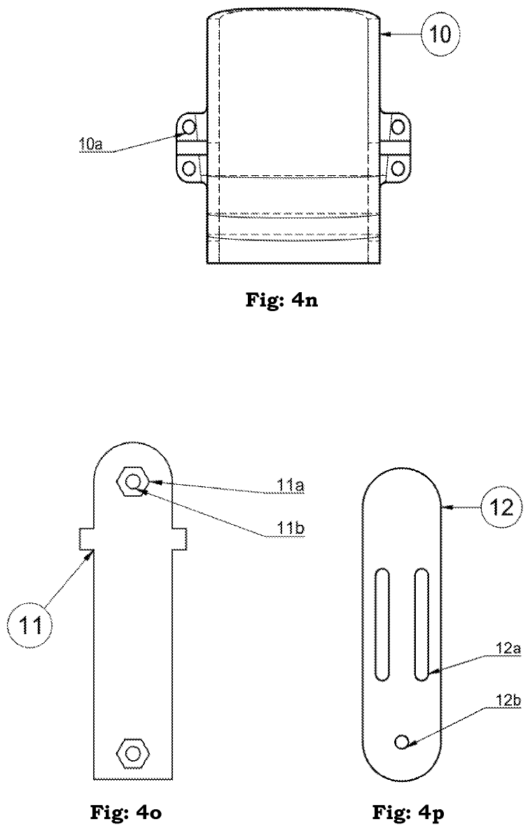

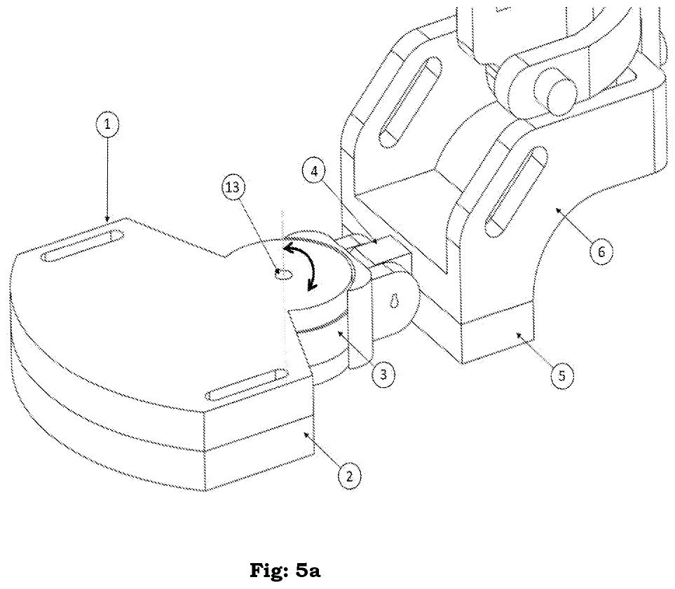

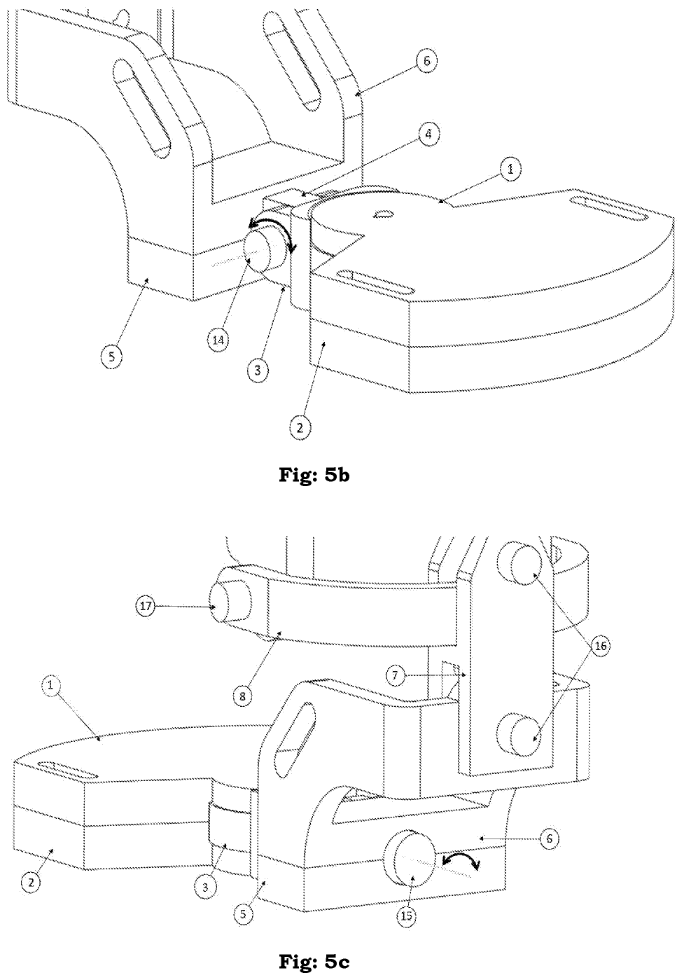

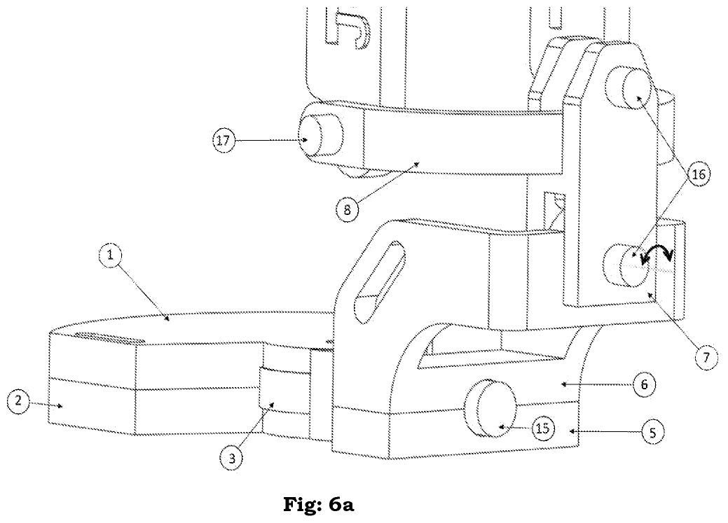

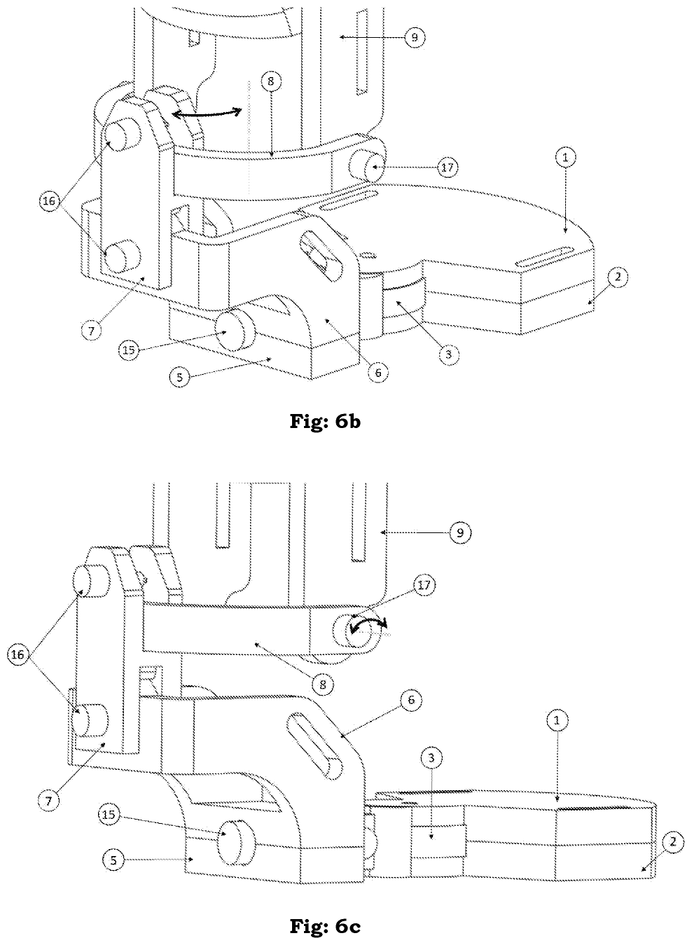

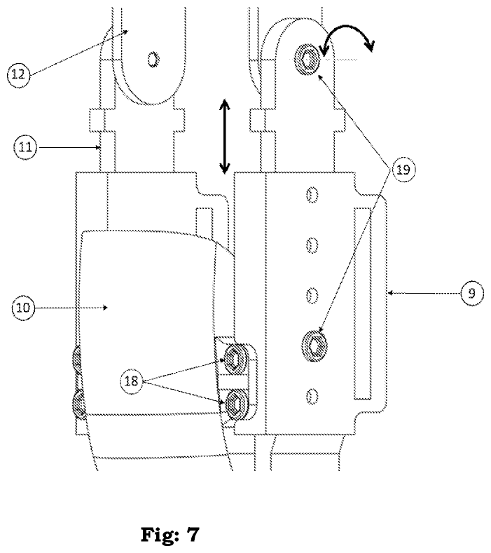

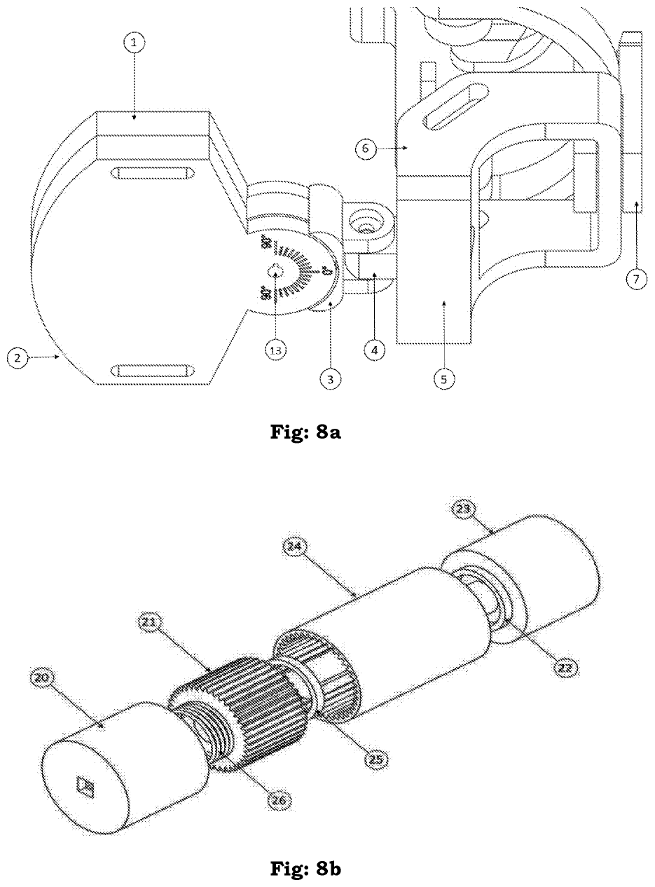

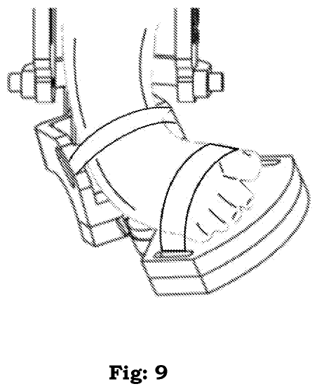

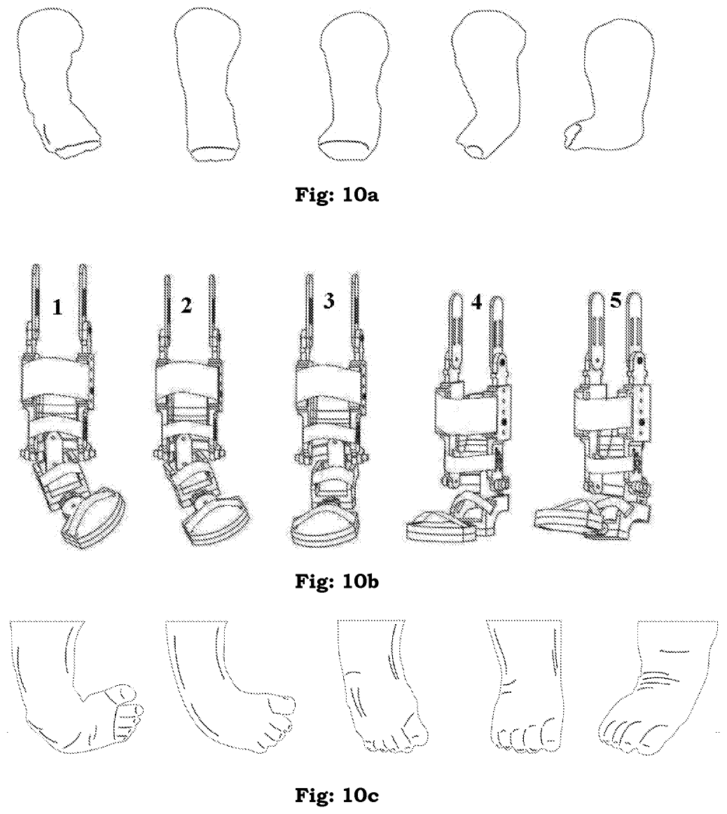

FIG. 4(k) shows: the Back and side cross-sectional view of Yaw Guide Base (7) with hole (7a and 7b).

FIG. 4(l) shows: the Side view of Hindfoot Yaw Link (8) with hole (8a).

FIG. 4(m) shows: the Side and cross-sectional view of Calf Support Link (9) with holes (9a, 9b, 9c, 9d) and cut sections (9e and 9f).

FIG. 4(n) shows: the Front view of Calf Muscle Support (10) with hole (10a).

FIG. 4(o) shows: the Side view of Slider Link (11) with hole (11a) and cut section (11b).

FIG. 4(p) shows: the Side view of Thigh Link (12) with cut section (12a) and hole (12b).

FIG. 5 collectively shows: the Orthosis supporting the forefoot motions in accordance with the present invention.

FIG. 5(a) shows: the forefoot Rolling (Inversion/Eversion).

FIG. 5(b) shows: the forefoot Yawing (Abduction/Adduction).

FIG. 5(c) shows: the forefoot Pitching (Dorsiflexion/Plantarflexion).

FIG. 6 collectively shows: the Orthosis supporting the hindfoot motions.

FIG. 6(a) shows: the hindfoot Rolling (Inversion/Eversion).

FIG. 6(b) shows: the hindfoot Yawing (Abduction/Adduction).

FIG. 6(c) shows: the hindfoot Pitching (Dorsiflexion/Plantarflexion).

FIG. 7 shows: the sliding motion of Slider Links and Rotation of Thigh Links in the orthosis of the present invention.

FIG. 8 shows: the locking and unlocking arrangements in Orthosis for corrective motions.

FIG. 8(a) shows: the Bottom view of forefoot yawing motion with dial gauge.

FIG. 8(b) shows: the mechanism for step motion actuation in Orthosis for corrective motions.

FIG. 9 shows: a severely deformed foot inside the developed corrective orthosis.

FIG. 10 collectively shows: the Corrective orthosis motions being performed similar to casting in the Ponseti method.

FIG. 10(a) shows: the cast for foot correction in succession as per Ponseti method.

FIG. 10(b) shows: the foot correction position of corrective orthosis in succession similar to casts in Ponseti method.

FIG. 10(c) shows: the foot inside the cast as well as orthosis in successive correction.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION WITH REFERENCE TO THE ACCOMPANYING DRAWINGS OF PREFERRED EMBODIMENTS OF THE INVENTION

While the embodiments of the disclosure are subject to various modifications and alternative forms, specific embodiment thereof have been shown by way of example in the figures and will be described below. It should be understood, however, that it is not intended to limit the disclosure to the particular forms disclosed, but on the contrary, the disclosure is to cover all modifications, equivalents, and alternative falling within the scope of the disclosure.

The present invention is directed to an invention pertinent to Orthosis for clubfoot (CTEV) Correction and Maintenance based on Ponsiti Method.

The developed orthosis is based on the Dr. Ponseti's understanding of foot biomechanics and Ponseti method of CTEV treatment. It allows the calcaneus to be free from any reaction force and a provision to hold the talus head as fulcrum during manipulations. This allows hindfoot to follow forefoot in a natural manner similar to Ponsiti method. The developed orthosis (FIG. 4) of the present invention incorporates a total of eight motions, out of which first six are corrective motions, three at forefoot level and three at hind foot level. Out of the other two motions, one caters to the growth of leg of the patient and another one knee movement of the patient.

The lock and unlock provisions along with dial gauges to all the six corrective motion mechanisms permits the clinicians to manipulate and hold various parts of the foot for successive corrections. The close loop actuators along with sensors helps in data recording by the controller cum data acquisition system and hence deformity measurement and database creation before, during and after the treatment of the patient can be automated.

The product of the proposed application is an externally applied brace (orthosis) to modify the structural and functional characteristics of the defective feet. It aims at correcting CTEV based on the Ponseti Method. The Ponseti method based orthosis for CTEV (clubfoot) correction and maintenance comprises of rigid links/parts (FIG. 4) to form various motions.

Now, reference may be made to the accompanying FIG. 4. The orthosis includes Metatarsal Top Link (1) superimposed on Metatarsal Bottom Link (2), each of which is provided with a slot (1b and 2b) respectively so as to hold foot as shown in FIGS. 4 (e) and (f). There is provision of Pitch and yaw link (3) having rotating joint with Metatarsal Top Link (1) and Metatarsal Bottom Link (2) to allow yawing of the forefoot using actuator A1 (13).

An ‘actuator’ here is an assembly of a dial gauge for a specified motion and its locking/unlocking mechanism.

The metatarsal top (1) and metatarsal bottom (2) are provided with congruent strap slots (1b,2b) allowing passage of strap through the slots (1b,2b) for holding baby foot steadily after manipulations by means of strap. The metatarsal top (1) and metatarsal bottom (2) are having keyholes (1a, 2a) for the actuator A1 (13) to pass through at the centre of the mating circular surface. These metatarsal top and bottom (1, 2) are coupled with pitch and yaw link (3) supporting the metatarsals to perform the forefoot yawing motion buoyed by the actuator A1 (13) through the hole (3a). Thus, the metatarsal top (1), metatarsal bottom (2) and pitch and yaw link (3) are connected together by the actuator A1 (13) with the pitch and yaw link (3) placed between the metatarsal top (1) and metatarsal bottom (2). The actuator A1 (13) passes through the holes (1a, 2a, 3a) as described.

The pitch and yaw link (3) is held in place and connected to the forefoot roll link (4) by actuator A2 (14) passing through the keyholes on both the linkages (3b and 4a) respectively as indicated in FIGS. 4 (g) and (h). The forefoot roll link (4) is further crammed in/connected to the upper and top bases of hind foot assembly (5, 6) to execute the rolling motion of the forefoot.

The Forefoot Roll Link (4) is having rotating joint with Pitch & Jaw Link (3) which gives the pitching motion of the forefoot through actuator A2 (14). The Hindfoot Top Base (6) is fixed with Hindfoot Bottom Base (5) and the Forefoot Roll Link (4) having cylindrical joint with both (5, 6) to provide rolling of the forefoot using actuator A3 (15). Here, the Hindfoot Top Base (6), Hindfoot Bottom Base (5) and Forefoot Roll Link (4) are connected to each other via said actuator A3 (15).

The hindfoot assembly encompasses hindfoot lower base (5), hindfoot top base (6) and a yaw guide base (7). The hindfoot top base (6) is principally made to support the talus. It positions the heel and entails the strap slots (6c). There is a substantially cylindrical cavity (5a, 6b) as in FIGS. 4 (i) and (j) flanked by the two hindfoot bases (5,6) which shelter the forefoot roll link (4). According to the present invention, the forefoot rolling motion is controlled by the actuator A3 (15) which locks the forefoot roll link (4) and thus, provides a clean and intricate structure, and effective functionality to the orthosis of present invention for the particular motion.

Yaw Guide Base (7) slides on the Hindfoot Yaw Link (8) to provide yawing of the hindfoot through actuator A4 (16). Yaw Guide Base (7) has a rotating joint with the Hindfoot Top Base (6) to provide rolling of the hindfoot through actuator A4 (16).

The yaw guide base (7) has two keyholes (7a), the lower one of which grips the hindfoot top base (6) and directs the hindfoot rolling motion using the actuator A4 (16); the upper hole (7a) clasps the hindfoot yaw link (8) through the actuator A4 (16). The upper actuator (A4 (16)) is responsible to uphold the hindfoot yawing motion and its locking based on the sliding of the Yaw Guide Base (7) slides on the Hindfoot Yaw Link (8) through actuator A4 (16).

The Calf Support Links (9) as illustrated in FIG. 4(m) are attached to the Hindfoot Jaw Link (8) through a rotating joint through holes (9d). This joint facilitates the pitching motion of the hindfoot using actuator A5 (17). The slot (9e & 90 are provided for straps to hold the leg.

The hindfoot yaw link (8) is suspended on calf support links (9) on two sides by actuator A5 (17) through holes on both the links (8a, 9d) respectively. This accomplishes the hindfoot pitching motion.

Calf Muscle Support (10) is provided to support the calf muscle and is fixed to the Calf Support Links (9) using fastener including Bolt (8) passing through holes (9a & 10a).

The calf support link is a comparatively complex part with a provision of intermittent support for different leg lengths. This is achieved by making multitude of holes (9b) on the calf support link (9) at different levels/heights to be locked with the slider link (11) using a slider bolt (19). The slider link (11) has a pair of embedded nuts and bolt holes (11a, 11b) for the same. The calf support link also has featured slots (9e, 90 to draw straps through them to tie around the leg.

The Slider Links (11) are attached to Calf Support Links (9) hole (9b) using Slider Bolt (19). Calf Support Links (9) have a plurality of holes (9b), which allows the Slider Links (11) to have linear motion inside Calf Support Links (9) to compensate for the growth of the leg during the treatment.

The Calf Muscle Support (10) is designed to support the calf muscle. It has protrusions towards the right and left with holes (10a) therein. As seen in the sectional view (E1-E1), there is an alternative pair of fasteners [nuts and bolt] holes (9c) on one side of the calf support link (9). These holes (9c) hold up the calf muscle support (10) in position using manifold bolts (18) passing through the holes (10a) in protruded parts of the calf muscle support (10).

The orthosis also includes two Thigh Links (12) for attaching and grounding of the orthosis with the patient thigh using straps passing through slot (12a) as shown in FIG. 4(p). The Thigh Links (12) replicate the above-knee part of the cast. These Thigh Links (12) are connected with two Slider Links (11) through a rotating joint. For this the Bolt (18) passes between two holes (11a and 12b).

The thigh link (12) has a minimal structure with a hole (12b) to attach the slider link (11) on one side using a slider bolt (19). Its function is to support the thigh even when the knees are bent. This link has a pair of slots (12a) for straps to be secured around the thigh.

Orthosis motions: The orthosis is capable to have motions required as per Ponseti method. The six motions at foot level are sufficient to cater the manipulations required as per the Ponseti Method. These motions occur in a minimum step size of 5 degrees as per requirement of the clinician.

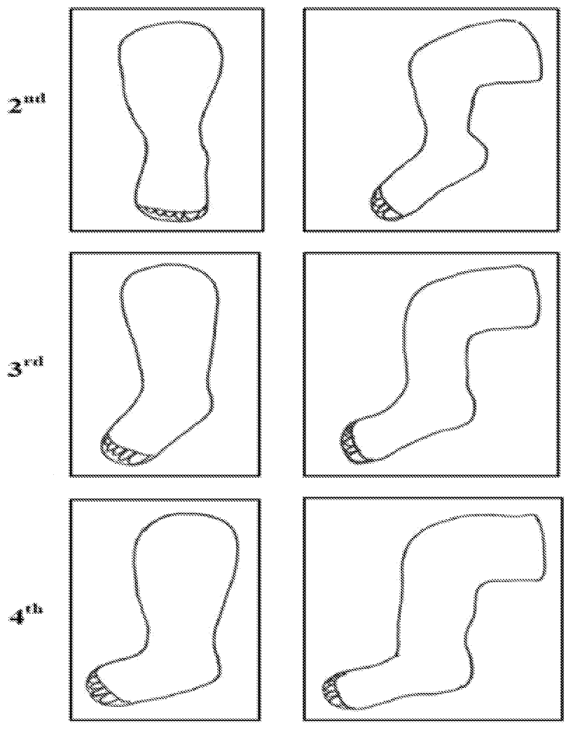

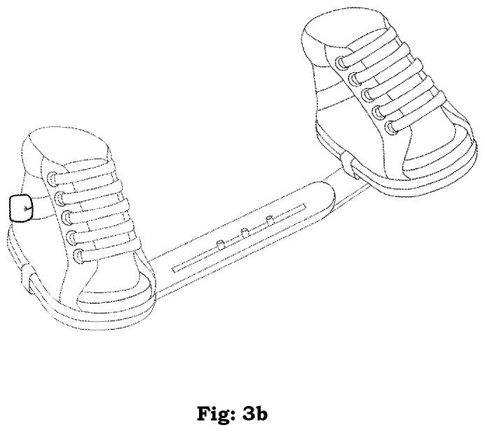

The abduction, eversion and dorsiflexion of the forefoot are handled by respective three motions in orthosis at forefoot level, as shown in FIG. 5. For the forefoot yawing, actuator A1 (13) actuates and gives a motion of 70-degree each in adduction as well as abduction, as shown in FIG. 5(a). The forefoot pitching occurs with the actuation of actuator A2 (14) with a range of 70-degree plantarflexion and 20-degree dorsiflexion, as indicated in FIG. 5(b). The forefoot rolling is handled by actuator A3 (15) actuation, with a range of 70-degree inversion and 20-degree eversion, as shown in FIG. 5(c). All the motions are with reference to FIG. 2(d).

The three motions at the hindfoot level give the corresponding response to these motions, which allows the hindfoot to follow forefoot, as shown in FIG. 6. The actuator A4 (16) provides rolling as shown in FIG. 6(a) with a range of 70-degree inversion and 20-degree eversion. The actuator A4 (16) also provides yawing of hindfoot as shown in FIG. 6(b), a motion of 70-degree each in adduction as well as abduction. The pitching of the hindfoot is carried out through actuator A5 (17), with a range of 20-degree plantarflexion and 70-degree dorsiflexion, as indicated in FIG. 6(c).

Apart from six corrective motions, the orthosis supports two other motions. The linear (sliding) motion in FIG. 7 includes sliding of Slider Links (11) in Calf Support Link (9) with adjustment of Bolt (19) at desired locations. This motion helps the growth of the leg of the patient during the treatment. Using the revolute joint (FIG. 7), concerning Bolt (19), the Slider Links (11) and Thigh Links (12) can be locked at any desired angle between zero to 90 degree to cater the folding and unfolding of patient's leg along with his/her knee.

Step Motion Actuation & Locking/unlocking Mechanism: The lock and unlock provisions along with dial gauges to all the six corrective motion mechanisms permits the clinicians to manipulate and hold various parts of the foot for successive corrections. Out of the six motions shown in FIG. 5 and FIG. 6, a case of such arrangement is shown in FIG. 8 for forefoot yawing (Abduction/Adduction) motion represented in FIG. 5(a). FIG. 8(a), shows the forefoot yawing (Abduction/Adduction) motion mechanism looking from bottom. The dial gauge has been provided around the cavity in Metatarsal Bottom part as shown in FIG. 4 (f). To activate the forefoot yawing motion in step of 5 degrees, and it's locking/unlocking, the actuator A1, assembly (13) with dial gauge involves a closed loop controlled electro-magnetic actuation mechanism and with sensor as shown in the FIG. 8(b).

For all the six corrective motions, by the actuators A(1-5) (13-17), the orthosis mechanism relies on indexing the rotary motion and locking and unlocking it using splines creates on outer body (24), inner slider (21) and outer cap (20). The slider in its initial position rests midway between outer cap (20) and outer body (24) making the splines interlock the outer cap (20) and outer body (24) connected to components that rotate relative to one another. Upon activation of electromagnet marked (22) the permanent magnet marked (25) attached permanently to inner body (21) is repelled towards the outer cap (20) allowing for rotation of outer cap (20) facilitated by motor (23). Once the required amount of rotation is achieved, the electromagnet (22) is deactivated and the spring (26), attached to both outer cap (20) and inner slider (21) restores the inner slider (21) returns to its position of rest locking the mechanism in place till further manipulation is required.

However, in case of manual operation an Allan key can be inserted into the cavity in the outer cap that pushes onto the inner slider unlocking the movement as it slides away from the outer cap allowing for required rotation and then restored to its original position as the spring (26) contracts bringing the inner slider (21) back to its position of rest locking it in place once again when the key is removed.

All the locking/unlocking mechanisms are devised to restrict any involuntary tempering with the orthosis while carrying the patient. The dial gauges allow the paramedical/parents to perform necessary manipulations through remote guidance.

Orthosis to follow deformed foot: The orthosis, through its mechanism follows the deformed foot to the extreme positions. The orthosis is designed to tackle both “benign”-minimum deformity and “severely deformed foot”-maximum deformity as described in Dimeglio Score. It implies that orthosis can handle the most deformed foot, in which the deformities can be upto 90 degrees, as shown in FIG. 9.

The close loop actuators along with sensors also helps in data recording by the controller cum data acquisition (DAQ) system and hence deformity measurement and database creation before, during and after the treatment of the patient can be automated. This provision of online continuous measurement and recoding avoids the need of additional clubfoot (CTEV) deformity measurement before classifying it. The recorded data in a central database paves the way for development of an expert system which can predict more customize, accurate and optimum treatment for patients.

Orthosis working for CTEV Correction and Maintenance: The orthosis allows the clinicians to correct the foot in the sequence as proposed in Ponseti method i.e. simultaneous correction of deformities as per foot bio-mechanics. Orthosis permits, Cavus correction by supinating the forefoot, adductus and varus deformities correction by the abduction of the forefoot with the lateral talus head as a fulcrum. After each manipulation, the orthosis is locked for 5 days to set the muscles. The graduated dial gauges permits the successive manipulations. With respect to the first stage of the Ponseti method, the manipulations required for cavus correction are handled by forefoot pitching and rolling. The adductus and varus correction is taken care of by forefoot yawing and pitching. FIG. 10, shows the corrective orthosis motions being performed as per the Ponseti method in succession replicating the steps of casting.

Once the CTEV is corrected, the orthosis needs to be worn by the patient unilaterally similar to the maintenance orthosis prescribed in Ponsiti method.

The orthosis also permits removal of residual equinus deformity at ankle joint through Achilles tenotomy, by the clinician, under local anesthesia, followed by locking the orthosis for 3 weeks. The orthosis has provision to provide 70 degree abduction and 15 degree dorsiflexion of complete foot similar to cast after tenotomy.

The orthosis is designed to cater various age ranges of the patient i.e. 2 weeks to 3 months, 3 months to 6 months, 6 months to 9 months, 9 months to 1 year and 1 year to 2 years, however the original aim is to start of the orthosis to the new born.

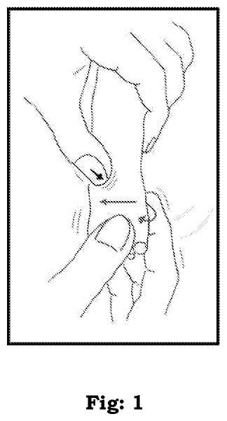

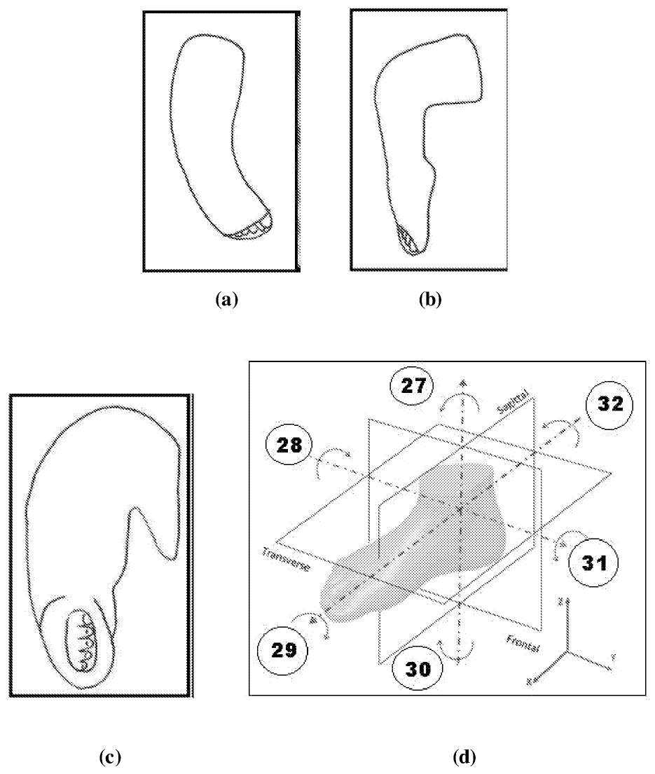

Design of the orthosis is parametric to customize it for the foot sizes of the patient. The orthosis also helps the clinician to identify the pivot point of talus head and lock the orthosis with respect to the pivot point for manipulation of the deformed foot for correction similar to FIG. 1.

The orthosis enables grounding it to the patient's leg through calf and thigh supports, hence making it unilateral. The motion at knee allows to replicate above knee cast as shown in FIG. 7. The orthosis permits knee movement from fully straightened to complete fold (FIG. 7), without any discomfort to the patient.

Through the mechanism of this orthosis, it can also be used for maintaining the corrected position of the foot unilaterally as the patient grows avoiding the chances of relapses. It is much easier and stigma free for parents and patients to use unilateral orthosis of instant invention as compared to bilateral orthosis of prior art.

The orthosis can also be employed for relapse cases both for correction and maintenance. The maintenance involves keeping the foot at 70 degree abduction, which can be easily obtained through yawing of forefoot. The orthosis is a unilateral device and looks like a high ankle shoe, thus avoiding the current stigma of bilateral FAB. The orthosis also supports the foot manipulation by informed parent and/or locally available para-medicos under the remote guidance of the clinician, minimizing patient travel to clinics, minimizing clinician time and encouraging treatment continuance.

As shown in FIG. 8, the corrective orthosis has provision of close loop actuator along with sensors for step motion as well as locking/unlocking mechanism. The orthosis can measure the CTEV deformity, initially for the assessment of the CTEV and later for quantifying the level of correction, every time the foot is manipulated with the help of integrated sensors.

Once the orthosis is mechanically established with direct operation by the clinicians and their trained people, it also supports remote foot manipulation by the clinician using close loop actuators and sensors for locking/unlocking on various joints along with controller and IoT device to the terminal at the clinician using software application. (FIG. 8). The sensors and actuators are placed along the lock and unlock provisions of the joint, along with dial gauges, which permits the autonomous manipulation of the orthosis for successive corrections using an algorithm based on Ponsiti's understanding of foot biomechanics and level of deformity of the foot. The sensor and actuator based manipulator operates under the instruction of a controller both guided and unguided through an intelligent algorithm.

The orthosis reduces the duration of overall treatment as the successive manipulations can be done after five days as the duration is sufficient for setting the muscles. The CTEV correction data can be stored in the big data base leading to development of AI based algorithms/expert system for CTEV correction. The orthosis paves the way for AI based clubfoot assessment and treatment upon development and training of algorithm by sufficient data sets in each category of clubfoot. The expert system can predict more customize, accurate and optimum treatment for patients, hence enhancing the overall clubfoot (CTEV) management experience at all stake holders level including patients, patents and clinicians.

### Test Results

All the orthosis dimensions and motions are first verified by using the principles of science, followed by Computer Aided Design, Free Body Diagrams, Computer Aided Engineering and Proof of Concept (PoC) developed. Additively manufactured PoC, using polylactide (PLA) was submitted to clinicians for inputs and corrections in orthosis is done. Minimum viable prototype (MVP) manufactured using carbon fiber reinforced nylon was further tested for its functionality and inputs from the clinicians and parents. Fully functional orthosis of size zero i.e. two week age was developed using additive manufacturing of continuous as well chopped carbon fiber reinforced nylon polymer. The feedback and results are highly encouraging. The product has the success rate that of Ponsiti Method i.e. at least 90% with all the advantages over the conventional Ponsiti Method.

### Advantageous Features of Orthosis:

It is based on the Ponsiti's understanding of foot biomechanics.
It incorporates eight motions namely, three corrective motions at forefoot level, three corrective motions at hind foot level, one motion to cater the growth of leg for the patient and one motion for knee flexibility of the patient.
The lock and unlock provisions along with dial gauges to all the corrective motion mechanisms permits the clinicians to manipulate and hold various parts of the foot for successive corrections.
Orthosis through its mechanism follows the deformed foot to the extreme positions
It allows the clinicians to correct the foot in the sequence as proposed in Ponsiti method i.e. simultaneous correction of all deformities, Cavus correction by supinating the forefoot, adductus and varus deformities correction by the abduction of the forefoot with the lateral talus head as a fulcrum, after each manipulation, the orthosis is locked for 5 days to set the muscles, the graduated dial gauges permits the successive manipulations
It also permits removal of residual equinus deformity at ankle joint through Achilles tenotomy by the clinician under local anaesthesia, followed by locking the orthosis for 3 weeks.
It is designed to cater various age ranges of the patient i.e. 2 weeks to 6 months, 6 months to 1 year and 1 year to 2 years, however the original aim is to start of the orthosis to the new born.
Design of the orthosis is parametric to customize it for the foot sizes of the patient.
It helps the clinician to identify the pivot point of talus head and lock the orthosis with respect the pivot point for manipulation of the deformed foot for correction.
It enables grounding it to the patient's leg through calf and thigh supports, hence rendering it unilateral.
It permits knee movement from fully straightened to complete fold.
Through the mechanism of this orthosis, it can also be used for maintaining the corrected position of the foot unilaterally as the patient grows avoiding the chances of relapses.
It can be used for relapse cases both for correction and maintenance.
It is a unilateral device and looks like a high ankle shoe, thus avoiding the current stigma of bilateral FAB.
It also supports the foot manipulation by informed parent and/or locally available para-medicos under the remote guidance of the clinician, minimizing patient travel to clinics, minimizing clinician time and encouraging treatment continuance.
It has provision of close loop actuator along with sensors for step motion as well as locking/unlocking mechanism.
It can measure the CTEV deformity, initially for the assessment of the CTEV and later for quantifying the level of correction, every time the foot is manipulated with the help of integrated sensors.
It supports remote foot manipulation by the clinician using actuators and sensors on various joints along with controller and IoT device to the terminal at the clinician using software application.
It reduces the duration of overall treatment as the successive manipulations can be done after five days as the duration is sufficient for setting the muscles.
It is much easier and stigma free for parents and patients to use unilateral orthosis of instant invention as compared to bilateral orthosis of prior art.
The CTEV correction data can be stored in the big data base leading to development of AI based algorithms/expert system for CTEV correction.
It paves the way for AI based clubfoot assessment and treatment upon development and training of algorithm by sufficient data sets in each category of clubfoot.
The developed expert system can predict more customize, accurate and optimum treatment for patients.

Following are the parts of the Orthosis: —

| Reference | |
|---|---|
| Numerals | Part Name |
| {1} | Metatarsal Top |
| {2} | Metatarsal Bottom |
| {3} | Pitch & Yaw link |
| {4} | Forefoot Roll link |
| {5} | Hindfoot Lower Base |
| {6} | Hindfoot Top Base |
| {7} | Yaw Guide Base |
| {8} | Hindfoot Yaw Link |
| {9} | Calf Support Link |
| {10} | Calf Muscle Support |
| {11} | Slider Link |
| {12} | Thigh Link |
| {13} | A1 (Assembly with dial gauge for forefoot yawing motion and |
| | its locking/unlocking) |
| {14} | A2 (Assembly with dial gauge for forefoot pitching motion |
| | and its locking/unlocking) |
| {15} | A3 (Assembly with dial gauge for forefoot rolling motion and |
| | its locking/unlocking) |
| {16} | A4 (Assembly with dial gauge for hindfoot yawing/rolling |
| | motion and its locking/unlocking) |
| {17} | A5 (Assembly with dial gauge for hindfoot pitching motion |
| | and its locking/unlocking) |
| {18} | Bolt |
| {19} | Slider Bolt |
| {20} | Outer Cap (splined internally) |
| {21} | Inner Slider (splined extrnally) |
| {22} | Electromagnet |
| {23} | Motor |
| {24} | Outer body (splined internally) |
| {25} | Permanent Magnet (attached permanently to inner slider) |
| {26} | Spring |
| {27} | Abduction (Yawing-in) |
| {28} | Dorsiflexion (Pitching-up) |
| {29} | Eversion (Rolling-CW from-Front) |
| {30} | Abduction (Yawing-out) |
| {31} | Plantarflexion (Pitching-down) |
| {32} | Inversion (Rolling-CCW from Front) |

### PRIOR ART REFERENCES

1. Faraj A A, Howorth C A, Nevelos A B. The use of adjustable hinged orthosis in treating talipes equino-varus. Foot and ankle surgery. 2004; 10(2):57158.
2. Lugo-Villeda L I, Frisoli A, Bautista F C, et al. Non-invasive biomechanical device for the club-foot medical treatment: A robotic rehabilitation analysis. In: 2008 2nd IEEE RAS & EMBS International Conference on Biomedical Robotics and Biomechatronics; IEEE; 2008. p. 323-328.
3. Khas K S, Pandey P M, Ray AR. Development of an orthosis for simultaneous three dimensional correction of clubfoot deformity. Clinical Biomechanics. 2018; 51:67-75 and Patent application number, 1336/DEL/2012 titled “Orthosis for Simultaneous Three-Dimensional Correction of Club Foot”
4. Mohamaddan S, Fu C S, Rasit A H, et al. Development of adjustable foot corrective device for clubfoot treatment. Pertanika Journal of Science & Technology. 2017; 25:251-258.
5. Vishnu V A, et. al. Design and development of orthosis for clubfoot correction in infants an additive manufacturing approach. Materials Today: Proceedings. 2019.
6. Mali H S, et al. “Development of remedial orthosis for ctev (clubfoot) patient through five corrective motions” Application no. 201811028509 [http://ipindiaservices.gov.in/PatentSearch/PatentSearch/NiewApplicationStatus]; 2018 [Accessed 26 May 2020].

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particulars claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogues to “at least one of A, B and C, etc.” is used, in general such a construction is intended in the sense one having skill in the art would understand the convention (e.g., “a system having at least one of A, B and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

The above description does not provide specific details of manufacture or design of the various components. Those of skill in the art are familiar with such details, and unless departures from those techniques are set out, techniques, known, related art or later developed designs and materials should be employed. Those in the art are capable of choosing suitable manufacturing and design details.

The terminology used herein is for the purpose of describing particular embodiments only and is not intended to be limiting of the present disclosure. It will be appreciated that several of the above-disclosed and other features and functions, or alternatives thereof, may be combined into other systems or applications. Various presently unforeseen or unanticipated alternatives, modifications, variations, or improvements therein may subsequently be made by those skilled in the art without departing from the scope of the present disclosure as encompassed by the following claims.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. Orthosis for clubfoot (CTEV) Correction and Maintenance based on Ponsiti Method comprising a metatarsal top (1), a metatarsal bottom (2) and a pitch and yaw link (3) connected together by an actuator A1 (13) with the pitch and yaw link (3) placed between the metatarsal top (1) and metatarsal bottom (2), wherein the metatarsal top (1) and metatarsal bottom (2) are provided with strap slots (1b,2b) allowing passage of strap through the slots (1b,2b) for holding baby foot;
the pitch and yaw link (3) is connected to forefoot roll link (4);
the forefoot roll link (4) is connected to upper and top bases of hind foot assembly (5, 6) to execute the rolling motion of the forefoot;
Yaw Guide Base (7) has a rotating joint with the Hind foot Top Base (6) to provide rolling of the hind foot through actuator A4 (16);
the Yaw Guide Base (7) slides on Hind foot Yaw Link (8) to provide yawing of the hind foot through the actuator A4 (16);
the hindfoot yaw link (8) is suspended on calf support links (9) on two sides;
Calf Muscle Support (10) is provided to support the calf muscle and is attached to the Calf Support Links (9).

2. The Orthosis as claimed in claim 1, wherein the Metatarsal Top Link (1) is superimposed on Metatarsal Bottom Link (2), each of which is provided with a slot (1b and 2b) respectively so as to hold foot, wherein the metatarsal top and bottom (1, 2) are coupled with pitch and yaw link (3) supporting the metatarsals to perform the forefoot yawing motion buoyed by the actuator A1 (13) through the hole (3a), in which the actuator A1 (13) passes through the holes (1a, 2a,3a).

3. The Orthosis as claimed in claim 1, wherein the pitch and yaw link (3) is held in place and connected to the forefoot roll link (4) by actuator A2 (14) passing through the keyholes on both the linkages (3b and 4a) respectively.

4. The Orthosis as claimed in claim 1, wherein the Forefoot Roll Link (4) is having rotating joint with Pitch and Jaw Link (3) which gives the pitching motion of the forefoot through actuator A2 (14), in which the Hindfoot Top Base (6) is secured with Hindfoot Bottom Base (5) and the Forefoot Roll Link (4) having joint with both bottom and top base (5,6) to provide rolling of the forefoot using actuator A3 (15), wherein the Hindfoot Top Base (6), Hindfoot Bottom Base (5) and Forefoot Roll Link (4) are connected to each other via said actuator A3 (15).

5. The Orthosis as claimed in claim 1, wherein the hind foot assembly encompasses hind foot lower base (5), hind foot top base (6) and a yaw guide base (7), in which the hind foot top base (6) is provided to support the talus.

6. The Orthosis as claimed in claim 1, wherein Yaw Guide Base (7) has a rotating joint with the Hindfoot Top Base (6) to provide rolling of the hindfoot through actuator A4 (16).

7. The Orthosis as claimed in claim 1, wherein the Yaw Guide Base (7) slides on the Hindfoot Yaw Link (8) to provide yawing of the hindfoot through actuator A4 (16).

8. The Orthosis as claimed in claim 1, wherein the hindfoot yaw link (8) is suspended on calf support links (9) on two sides by actuator A5 (17) through holes on both the links (8a, 9d) respectively to accomplish the hindfoot pitching motion.

9. The Orthosis as claimed in claim 1, wherein Calf Muscle Support (10) is provided to support the calf muscle and is fixed to the Calf Support Links (9) using fastener including Bolt (18) passing through holes (9a & 10a), multitude of holes (9b) are provided on the calf support link (9) at different levels/heights to be locked with the slider link (11) using a slider bolt (19), in which the slider link (11) has a pair of embedded nuts and bolt holes (11a, 11b) for the same and the calf support link has slots (9e, 9f) to draw straps there through to tie around the leg.

10. The Orthosis as claimed in claim 1, wherein the Slider Links (11) are attached to Calf Support Links (9) having a plurality of holes (9b), which allows the Slider Links (11) to have linear motion inside Calf Support Links (9) to compensate for the growth of the leg during the treatment.

11. The Orthosis as claimed in claim 1, wherein the Calf Muscle Support (10) supports the calf muscle, which is having protrusions towards the right and left with holes (10a) therein.

12. The Orthosis as claimed in claim 1, wherein the orthosis includes thigh Links (12) for attaching and grounding of the orthosis with the patient thigh using straps passing through slot (12a), wherein the Thigh Links (12) replicate the above-knee part of the cast and the Thigh Links (12) are connected with two Slider Links (11) through a rotating joint, for which a fastener including Bolt (18) passes between two holes (11a and 12b).

13. The Orthosis as claimed in claim 1, wherein the thigh link (12) has a hole (12b) to attach the slider link (11) on one side using a slider bolt (19), that supports the thigh even when the knees are bent, in which the thigh link (12) has a pair of slots (12a) for straps to be secured around the thigh.

14. The Orthosis as claimed in claim 1, wherein the orthosis involves eight motions, out of which first six are corrective motions, three at forefoot level and three at hind foot level and out of the other two motions, one caters to the growth of leg of the patient and another one knee movement of the patient.

15. The Orthosis as claimed in claim 1, wherein the orthosis mechanism relies on indexing the rotary motion and locking and unlocking it using splines creates on outer body (24), inner slider (21) and outer cap (20), in which the slider in its initial position rests midway between outer cap (20) and outer body (24) making the splines interlock the outer cap (20) and outer body (24) connected to components that rotate relative to one another, wherein Upon activation of electromagnet marked (22) the permanent magnet marked (25) attached permanently to inner body (21) is repelled towards the outer cap (20) allowing for rotation of outer cap (20) facilitated by motor (23), wherein Once the required amount of rotation is achieved, the electromagnet (22) is deactivated and the spring (26), attached to both outer cap (20) and inner slider (21) restores the inner slider (21) returns to its position of rest locking the mechanism in place till manipulation is required.

16. The Orthosis as claimed in claim 1, wherein in case of manual operation an Allan key is inserted into the cavity in the outer cap that pushes onto the inner slider unlocking the movement as it slides away from the outer cap allowing for required rotation and then restored to its original position as the spring (26) contracts bringing the inner slider (21) back to its position of rest locking it in place when the key is removed.

17. The Orthosis as claimed in claim 1, wherein the corrective orthosis has provision of close loop actuator along with sensors for step motion as well as locking/unlocking mechanism, leading to automated deformity measurement, database creation and expert system development, which can predict more customize, accurate and optimum treatment for patients
